(19) United States
(12) Patent Application Publication
(10) Pub. No.: US 2015/0044304 A1
(43) Pub. Date: Feb. 12, 2015

(54) COMPOSITION FOR IMPROVING NEUROPSYCHOLOGICAL TEST BATTERY SCORE

(71) Applicant: N.V. Nutricia, Zoetermeer (NL)

(72) Inventors: Martine Groenendijk, Barendrecht (NL); Anke Bongers, Veenendaal (NL); Patrick Joseph Gerardus Hendrikus Kamphuis, Utrecht (NL)

(21) Appl. No.: 14/355,176

(22) PCT Filed: Oct. 30, 2012
(86) PCT No.: PCT/NL2012/050754

§ 371 Date: Apr. 29, 2014

(30) Foreign Application Priority Data
Oct. 31, 2011 (NL) NL2011/050739
Nov. 4, 2011 (EP) 11187958.1

(51) Int. Cl. A61K 31/7072; A61K 31/675; A61K 31/714; A61K 31/519; G09B 19/00; A61K 31/355; A61K 31/14; A61K 31/375; A61K 33/04; A61K 31/202; A61K 31/7068

(52) U.S. Cl. CPC A61K 31/7072; A61K 31/202; A61K 31/675; A61K 31/714; A61K 31/519; A61K 31/7068; A61K 31/355; A61K 31/14; A61K 31/375; A61K 33/04; G09B 19/00
USPC 424/702; 514/50; 434/156

(57) ABSTRACT

The invention relates to a composition comprising: (i) one or more of uridine and cytidine, or salts, phosphates, acyl derivatives or esters thereof; and (ii) a lipid fraction comprising at least one of docosahexaenoic acid (22:6; DHA), eicosapentaenoic acid (20:5; EPA) and docosapentaenoic acid (22:5; DPA), or esters thereof, for use in the improvement of the composite NTB score. The invention also provides an improved NTB method comprising an optimized set of individualized tests, in particular for a (prodromal or mild) Alzheimer's or dementia patient.

### FIELD OF THE INVENTION

The invention is in the field of treatment of neuropsychological disorders and more particularly relates to a composition for use in improving neuropsychological test battery, for subjects suffering from or at risk of developing memory disorder, memory decline and cognitive dysfunction. In one aspect, the invention particularly rests in providing ways to monitor the progress of cognitive function in said subjects.

### BACKGROUND DESCRIPTION

Neuropsychological disorders, including age-associated memory impairment (AAMI), Alzheimer's disease and dementia, play an important role in our society. While the clinical stages of those disorders are diagnosed at later stages, many aspects of those disorders, in terms of memory decline and loss of executive functions, manifest earlier in life. A schematic overview of the various stages is shown in FIG. 1. It is important to monitor these symptoms as early as possible, in order to optimize treatment.

To that end, neuropsychological tests have been developed, which tests involve specifically designed tasks used to measure a psychological function known to be linked to a particular brain structure or pathway. These tests usually involve the systematic administration of clearly defined procedures in a formal environment, and they form a core component of the process of conducting neuropsychological assessment, along with personal, interpersonal and contextual factors (source: http://en.wikipedia.org/wiki/Neuropsychological_test).

A broad spectrum of neuropsychological tests is availabe, and these can be organised into broad categories of memory, language, executive function and dementia specific testing, based on the cognitive function which they predominantly assess. After all, most forms of cognition (impairment) actually involve multiple cognitive functions working in unison. There are some test batteries which combine a range of such tests to provide an overview of cognitive skills. The surplus of these test batteries is that it combines these individual measures into an overall assessment designed to yield a single efficacy measure of cognitive changes.

Amongst these test batteries is the so called Neuropsychological Test Battery [NTB]. According to J. Harrison et al. “10 Years of the Neuropsychological Test Battery (NTB)” Patient Reported Outcomes Newsletter 46 (Fall issue 2011) 21-24, the NTB is a composite cognitive measure comprised of standardized tests that have been in use in the field of clinical psychology for, in some case, more than 60 years. All of the selected measures have been extensively individually validated. The elements of the ‘NTB standard’ employed in AN1792-201 have been summarized in Table 1. What distinguishes NTB from other traditionally employed measures, such as the mini-mental state examination [MMSE] or ADAS-cog, is its focus on i) associative learning and paradigm for assessing episodic memory, and ii) the assessment of executive functions [EF], such as planning, strategy and working memory. Measures of EF are recognized to be robustly correlated with instrumental activities of daily living, and it was considered worthwile to include EF measures into testing. Validation of the NTB in the field has been reviewed in more detail in the aforementioned Harrison et al. paper, which contents is considered herein incorporated by reference.

TABLE 1
Original composition of the NTB

| Test | domain | 1^{st} outcome | 2^{nd} outcome |
|---|---|---|---|
| Visual Paired Associates | memory | Immediate recall | Delayed recall |
| Verbal Paired Associates | Memory | Immediate recall | Delayed recall |
| Rey Auditory Verbal | Memory | Immediate recall | Delayed recall |
| Learning Test | | | |
| Controlled Oral | EF | Total number of | — |
| Word Association Test | | acceptable words | |
| Category Fluency Test | EF | Total number of | — |
| | | acceptable words | |
| Digit Span | EF | Sequences | — |
| | | correctly recalled | |

The NTB has now been used as a cognitive outcome measure in dementia clinical drug trials for more than 10 years. This period has witnessed changes in both the composition and administration of the NTB, as well as a good deal of further information with respect to its psychometric properties.

In the past, Prana Biotechnology developed a variant of the NTB, based on a preliminary suggestion to focus on procognitive effects. Consequently, the NTB content for the Prana study was changed to remove one test of memory, i.e. the Wechsler memory scale [WMS] Visual Paired Associates test (both immediate and delayed recall) which was replaced by a further test of executive function, the Trail Making Test [TMT]. Details are provided in L. Lannfelt L et al. Lancet Neurology 2008; 7(9):779-786.

Over the years, other versions of the NTB have been suggested. Of the six tests that comprise the original NTB, the EF elements are rarely altered. They are more often augmented, such as occurred in the aforementioned Prana trial.

In addition, there is an ongoing need for improving memory and executive function of a subject in need thereof, particularly finding ways to improve the NTB scores of a subject suffering from memory or cognitive disorder, memory decline or cognitive dysfunction.

### SUMMARY OF THE INVENTION

In one aspect, with regard to the aforementioned Prana study, the inventors have now improved the Neuropsychological Test Battery even further, particularly to render it reliable to assess changes in (prodromal or mild) Alzheimer's or dementia patients, in particular in a drug-naive (prodromal or mild) Alzheimer's or dementia patients, and/or subjects with a mini-mental state examination of 20-30.

To that end, the NTB amended in the Prana study has been amended to include an ADAS-cog orientation task test and a letter digit substitution test, directed at the memory and attention domain, respectively.

The ADAS-cog is the cognitive subscale of the Alzheimer's Disease Assessment Scale (ADAS), developed and validated for AD patients. Since the method and Neuropsychological Battery Test of the invention are primarily supposed to be sensitive in the early stage of AD and dementia, particularly prodromal or mild AD or dementia patients, it is only the orientation task of the ADAS-cog that is applied mandatorily. This task is designed to determine how well oriented the patient is with regard to time and place.

By carefully selecting the test methods to be comprised in the NTB, it enables the test administrators to focus on the mild and prodromal aspects of AD and dementia, while preferably keeping the time needed to assess cognition (including executive function) to less than 30 minutes.

In another aspect, the invention rests in providing a composition for improving the overall performance in terms of executive function and memory function, by providing a subject in need thereof with a composition comprising:

i) one or more of uridine and cytidine, or salts, phosphates, acyl derivatives or esters thereof; and
ii) a lipid fraction comprising at least one of docosahexaenoic acid (22:6; DHA), eicosapentaenoic acid (20:5; EPA) and docosapentaenoic acid (22:5; DPA), or esters thereof.

The invention particularly relates to a composition for use in improving the NTB global or composite score, taking into account executive function and memory function, and preferabyl also results obtained for ADAS-cog orientation task and letter digit substitution test.

### LIST OF FIGURES

FIG. 1 shows the different stages of cognitive decline in Alzheimer's Disease. Source: Sperling et al 2011;

FIG. 2 shows a schematic block diagram of an embodiment of a computer system 10;

FIG. 3 shows the NTB composite score including executive domain and memory domain scores, and the ADAS-cog orientation task and the letter digit substitution test (p=0.370); and

FIG. 4 shows the global NTB score based on executive domain and memory domain scores. The model is a second order polynomial (p=0.117).

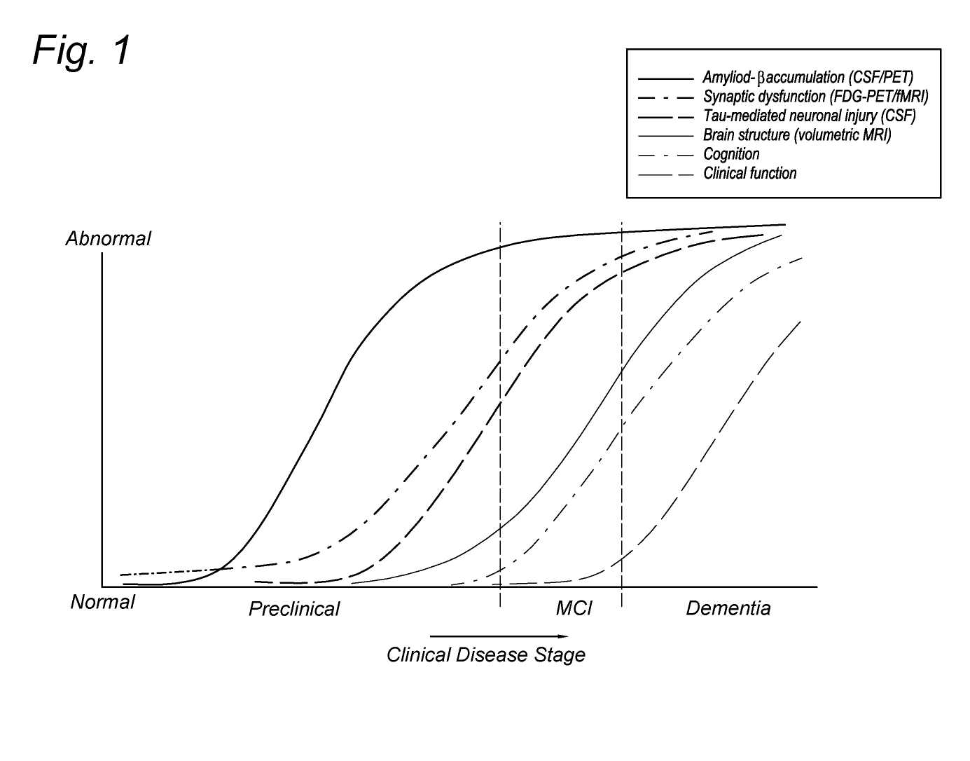

### LIST OF PREFERRED EMBODIMENTS

1. Use of a composition for the manufacture of a product for improving the composite Neuropsychological Test Battery [NTB] score of a subject in need thereof, wherein said composition comprises:
   i) one or more of uridine and cytidine, or salts, phosphates, acyl derivatives or esters thereof; and
   ii) a lipid fraction comprising at least one of docosahexaenoic acid (22:6; DHA), eicosapentaenoic acid (20:5; EPA) and docosapentaenoic acid (22:5; DPA), or esters thereof.
2. Use of a composition for the manufacture of a product for treating a subject in need thereof, wherein said composition comprises:
   i) one or more of uridine and cytidine, or salts, phosphates, acyl derivatives or esters thereof; and
   ii) a lipid fraction comprising at least one of docosahexaenoic acid (22:6; DHA), eicosapentaenoic acid (20:5; EPA) and docosapentaenoic acid (22:5; DPA), or esters thereof, and wherein said subject is subjected to an NTB.
3. Use according to embodiment 2, comprising calculating a composite score from the data retrieved for the individual tests of the NTB.
4. Use according to any one of the preceding embodiments, wherein said subject suffers from a memory or cognitive disorder, memory decline or cognitive dysfunction, such as Age Associated Memory Impairment (AAMI), Alzheimer's Disease, multiple sclerosis, vascular dementia, frontotemporal dementia, semantic dementia or dementia with Lewy bodies.
5. Use according to any one of the preceding embodiments, wherein said subject suffers from Alzheimer's Disease or dementia syndrome, including mild or prodromal AD or dementia.
6. Use according to any one of the preceding embodiments, wherein said subject has a mini-mental state examination (MMSE) of 20-30, preferably 20-26, more preferably 24-26.
7. Use according to any one of the preceding embodiments, wherein said composition comprises choline, or salts or esters thereof, preferably 200-600 mg choline per daily dose or per 100 ml composition.
8. Use according to any one of the preceding embodiments, wherein said composition comprises at least one, preferably at least two, most preferably all B vitamins selected from the group consisting of vitamin B6, vitamin B12 and vitamin B9.
9. Use according to any one of the preceding embodiments, wherein said composition comprises, per daily dose or preferably per 100 ml composition, at least 500 mg of DHA, preferably at least 600 mg of DHA, and at least 50 mg of uridine, preferably at least 100 mg of uridine.
10. Use according to any one of the preceding embodiments, wherein the composition comprises, per daily dose or preferably per 100 ml composition:
   50-1000 mg phospholipids,
   0.5-3 mg vitamin B6,
   50-500 μg folic acid,
   1-30 μg vitamin B12.
11. Use according to any one of the preceding embodiments, wherein the composition comprises, per daily dose or preferably per 100 ml composition:
   100-500 mg, preferably 200-400 mg EPA,
   1000-1500 mg, preferably 1100-1300 mg DHA,
   50-600 mg, preferably 60-200 mg phospholipids,
   200-600 mg, preferably 300-500 mg choline,
   400-800 mg, preferably 500-700 mg UMP (uridine monophosphate),
   20-60 mg, preferably 30-50 mg vitamin E (alpha-TE),
   60-100 mg, preferably 70-90 mg vitamin C,
   40-80 μg, preferably 50-70 μg selenium,
   1-5 μg, preferably 2-4 μg vitamin B12,
   0.5-3 mg, preferably 0.5-2 mg vitamin B6, and
   200-600 μg, preferably 300-500 μg folic acid.
12. Use according to any one of the preceding embodiments, wherein said NTB comprises at least 1, 2, 3, 4, 5, 6, 7, 8 or all of:
   a. Wechsler memory scale [WMS]—verbal paired associates test;
   b. Rey Auditory Verbal Learning test recall [RAVLT recall];
   c. Recognition test, preferably selected from the group consisting of:
      i. Rey Auditory Verbal Learning test [RAVLT recognition];
      ii. Repeatable Battery for the Assessment of Neuropsychological Status [RBANS];
      iii. California Verbal Learning Test [CVLT]; and
      iv. Consortium to Establish a Registry for Alzheimer Disease Test [CERAD];
   d. WMS digit span test;
   e. Controlled Word Association test [COWAT];
   f. Category fluency test;
   g. Trail Making Test [TMT];
   h. Orientation task ADAS-cog test; and
   i. Letter digit substitution test.
13. Use according to embodiment 12, wherein said RAVLT recall comprises at least one, preferably both of RAVLT immediate and delayed recall test.
14. Use according to embodiments 12 or 13, wherein said NTB comprises (b) RAVLT immediate recall and RAVLT delayed recall, and (c) RAVLT recognition.
15. Use according to any one of embodiments 12-14, wherein said NTB comprises:
   WMS VPA immediate test;
   WMS VPA delayed test;
   RAVLT immediate recall test;
   RAVLT delayed recall test;
   RAVLT recognition test;
and
   WMS digit span test;
   COWAT test;
   Category fluency test;
   TMT, preferably at least TMT B;
and optionally:
Orientation task ADAS-cog test; and Letter digit substitution test.
16. A method for assessing cognition of a subject, comprising subjecting said subject to:
a. Wechsler memory scale [WMS]—verbal paired associates test;
b. Rey Auditory Verbal Learning test recall [RAVLT recall];
c. Recognition test, preferably selected from the group consisting of:
   i. Rey Auditory Verbal Learning test [RAVLT recognition];
   ii. Repeatable Battery for the Assessment of Neuropsychological Status [RBANS];
   iii. California Verbal Learning Test [CVLT]; and
   iv. Consortium to Establish a Registry for Alzheimer Disease Test [CERAD];
d. WMS digit span test;
e. Controlled Word Association test [COWAT];
f. Category fluency test;
g. Trail Making Test [TMT];
h. Orientation task ADAS-cog test; and
i. Letter digit substitution test.
   and optionally comprising administering said subject with a composition aiming at improvement of cognitive function.
17. The method according to embodiment 16, wherein said RAVLT recall comprises at least one, preferably both of RAVLT immediate and delayed recall test.
18. Use according to embodiment 16 or 17, wherein said NTB comprises a (b) RAVLT immediate recall, RAVLT delayed recall and a (c) RAVLT recognition test.
19. The method according to any one of embodiments 16-18, comprising calculating memory function and executive function domain scores and/or calculating a composite score from the data retrieved for the individual tests (a)-(i).
20. The method according to embodiment 19, comprising comparing the data retrieved for the individual test scores (a)-(i), the calculated memory function and executive function domain scores and/or the calculated composite score at various points in time.

### DETAILED DESCRIPTION OF THE INVENTION

The inventors have observed that after administration of a product comprising (i) one or more of uridine and cytidine, or salts, phosphates, acyl derivatives or esters thereof, and (ii) a lipid fraction comprising at least one of docosahexaenoic acid (22:6; DHA), eicosapentaenoic acid (20:5; EPA) and docosapentaenoic acid (22:5; DPA), or esters thereof, both memory function and executive function can be improved, in particular in a Alzheimer's or dementia patient. This conclusion was the result of clinical trials involving many neuropsychological tests focusing on executive function and memory function.

In one aspect, the invention pertains to a method for assessing cognition of a subject, comprising subjecting said subject to:

a. Wechsler memory scale [WMS]—verbal paired associates test;
b. Rey Auditory Verbal Learning test recall [RAVLT recall];
c. Recognition test;
d. WMS digit span test;
e. Controlled Word Association test [COWAT];
f. Category fluency test;
g. Trail Making Test [TMT];
h. Orientation task ADAS-cog test; and
i. Letter digit substitution test.

In one embodiment, the method comprises administering said subject with a composition aiming at improvement of cognitive function (including memory and executive function), preferably involving at least daily administration. The composition or ‘drug’ may be a pharmaceutical or nutritional composition, and it may be a new or existing composition. It is preferred that the composition comprises (i) and (ii) as described here below, and optionally comprising one or more of the further characteristics discussed in the present application.

In a further embodiment, the method preferably comprises collecting data retrieved in said tests with a computer system. The term ‘collecting data’ is understood to encompass collecting at least the scores for the individual tests (a)-(i), and/or calculating domain scores from scores for the individual tests (a)-(i), and/or calculating a composite (global) score from scores for the individual tests (a)-(i). The method may further comprise comparing the individual test scores (a)-(i), the domain score and/or the composite (global) score of the subject subjected to the tests (a)-(i) at various times during the assessment or treatment.

The ‘assessment of cognition’ involves searching and detecting improvement or decline in memory and executive functions.

The term ‘assessing’ is construed to encompass monitoring said subject at different times over a period of at least 12 weeks, more preferably at least 13, 14, 15, 16, 17, 18, 19, 20, 21, 22, 23, most preferably at least 24 weeks. The assessment may preferably be carried out at least two, more preferably at least three times, most preferably at least four times during the above monitoring period. For monitoring, the tests are preferably carried out at regular intervals, e.g. every two or three weeks, in accordance with the standardized instructions for the individual tests. The assessment may be part of a treatment.

In a further aspect, the invention pertains to a computer system (carrier) comprising means for collecting individual test scores of a subject and/or means for computing a domain score based on individual test scores of a subject and/or means for calculating a composite (global) score of a subject, wherein said subject has been subjected to the above-mentioned individualized items a-i.

In yet a further embodiment, the invention pertains to a kit of parts comprising:

(i) instructions for the above-mentioned individualized items a-i, and
(ii) a composition aiming at improvement of cognitive function. The composition or ‘drug’ may be a pharmaceutical or nutritional composition, and it may be a new or existing composition.

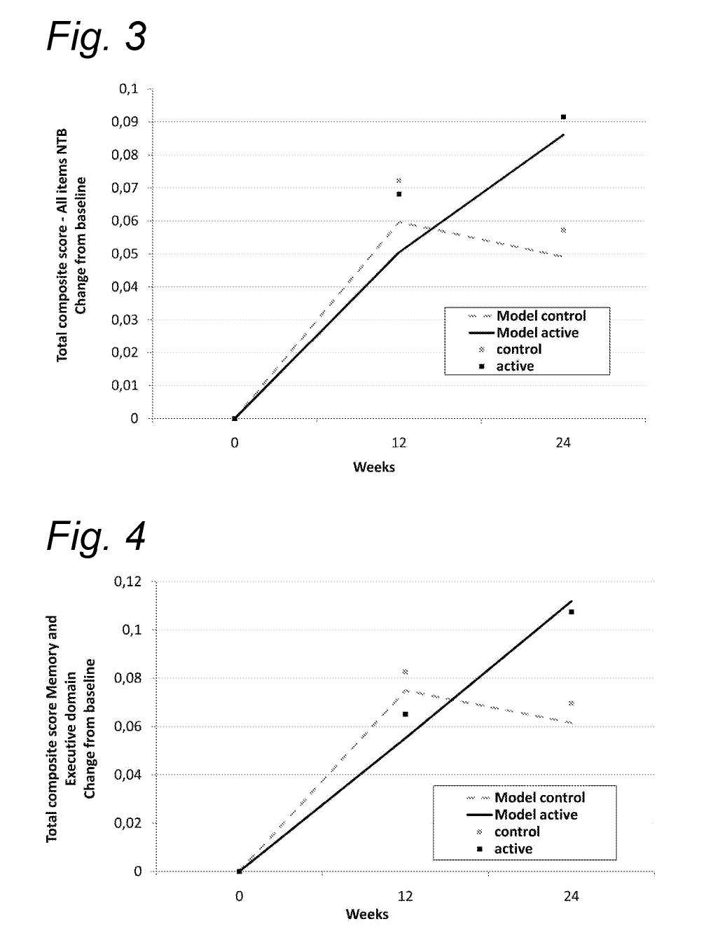

The inventors have surprisingly found that the composite NTB score of a subject is improved significantly when said subject is administered with a composition comprising (i) and (ii) as described here below. In the field, the global or composite NTB score is based on the results of the individualized tests. The results of the NTB tests (a), (b) and (c) may be collected in a so called ‘memory function domain score (z-score)’, and the results of the NTB tests (d)-(g) may be collected in a so called ‘executive function domain score (z-score)’. The definition for the memory function domain and the executive function domain are derived from the validated NTB disclosed in Harrison J, Minassian S L, Jenkins L, Black R S, Koller M, Grundman M. A neuropsychological test battery for use in Alzheimer disease clinical trials. Arch Neurol. 2007; 64(9):1323-1329, its contents herein considered incorporated by reference. The contributions from the individual NTB tests to the domain score have been studied by performing a factor analysis that has now become established practice. This factor analysis is to be applied in the calculation of the domain z-score here. The NTB composite score may—in addition to the contributions of the memory and executive function—also incorporate the test results of the individual tests (h) and (i). This is for instance shown for the clinical study presented herein, the results in terms of composite NTB score depicted in FIGS. 3 and 4.

Throughout the application, the terms ‘global NTB score’, ‘composite NTB score’, ‘NTB global score’ and ‘NTB composite score’ are used interchangeably. The memory function domain score is preferably based on WMS VPA immediate test; WMS VPA delayed test; RAVLT immediate recall test; RAVLT delayed recall test; and RAVLT recognition test.

The executive function domain score is preferably based on WMS digit span test; COWAT test; Category fluency test; TMT, preferably at least TMT B.

The global score is preferably based on the composite of the above memory function domain score and the executive function domain score, and optionally further comprises Orientation task ADAS-cog test; and Letter digit substitution test.

In a further aspect, the invention pertains to the use of a composition comprising (i)-(ii) and optionally (iii) as defined above in the manufacture of a product for treating a subject in need thereof, and subjecting said subject to a NTB, preferably comprising at least 2, 3, 4, 5, 6, 7, 8 or all of the aforementioned tests (a)-(i). The composition is preferably administered to said subject at least on daily basis. The method preferably involves monitoring said subject with a NTB, preferably comprising at least 2, 3, 4, 5, 6, 7, 8 or all of the aforementioned tests (a)-(i) during the treatment.

The method or use of the invention comprises administering the composition comprising the aforementioned ingredients, and as further outlined below, to a subject in need thereof. The prophylactic or preventive aspect includes reducing the risk of occurring of the disorders.

The treatment preferably involves daily administration of the product, preferably for at least 12 weeks. The product is preferably administered (daily) for at least 13 weeks, more preferably at least 14, 15, 16, 17, 18, 19, 20, 21, 22, 23, most preferably at least 24 weeks. With ‘improvement’ it is intended to mean that the composite score increase compared to a control group of subjects suffering from the same condition but not given the composition of the invention.

The composite NTB score is preferably based on at least two individual tests from the aforementioned list (a)-(i), all of which are standardized in the art. The NTB preferably comprises at least one test directed at the executive function domain, and at least one test directed at the memory function domain. The NTB preferably comprises at least 2, more preferably at least 3 tests for the EF domain and at least 2, more preferably at least 3 tests for the memory domain. Most preferably, the NTB comprises at least 6, more preferably at least 7, most preferably comprises 8, even more preferably comprises 9, most preferably consists of all of the tests of the aforementioned list (a)-(i). In one embodiment, the composite NTB score is calculated from (a)-(g). In another embodiment, the composite NTB score is calculated from (a)-(g) and (h) and (i). As demonstrated in the experimental section, in both cases improvements have been observed when administered the composition of the invention.

It is not part of the invention to amend the known individual tests (a)-(i). As a guide, the tests are outlined here below. However, the instructions given here below are by no means regarded to be limiting to the invention.

### Wechsler Memory Scale—Verbal Paired Associates Test (Immediate and Delayed Recall)

Paired associative learning tests require the subject to associate two items in memory. Some tasks that feature this paradigm require the subjects to associate colours with shapes or objects with spatial locations. In the field, Wechsler memory scale—visual test (immediate and delayed recall) is abbreviated as ‘WMS VPA immediate’ and ‘WMS VPA delayed’, respectively. The VPA requires the subjects to associate two words in memory.

In the VPA test, the subject is read out loud a number of word pairs, preferably about 8. The word pairs involve some (e.g. 4) ‘easy’ pairs that are semantically related, such as ‘BABY—CRIES’ or ‘METAL—IRON’ and some (e.g. 4) ‘Hard’ pairs that require an association to be made between semantically unrelated word pairs, such as ‘CRUSH—DARK’. The word pairs are presented auditorily and then immediate recall is tested by having the test administrator say the first word of each pair as a cue. The subjects must in response to the cue say the paired word. This is carried out a number of times, preferably for 3-6 groups of word pairs, dependent on patient's performance. The patient is awarded one point for each correct response.

The test includes a delayed recall condition, e.g. 1 to 30 minutes, after the immediate recall test (WMS-VPA delayed) and in an attempt to encourage high levels of delayed recall, study participants who fail to score the highest score (e.g. 8 on trial 3) of immediate recall then undergo further trials (e.g. a 4th, 5th and, if necessary, 6th trial). If perfect performance is achieved, e,g on trial 3 or any successive trial, the immediate recall component is ended. If not, further trials are administered (e.g. to a maximum of six trials). At delayed recall all cue words (e.g. 8) are presented and the subjects are requested to provide the correct paired word. Immediate recall yields a score comprised between 0 and pairs number×trials (e.g. between 0 and 8 pairs×3 trials=24) and delayed recall a score comprised between 0 and the number of pairs (e.g. 0-8). In both cases higher scores indicate superior performance.

Reference is made to Wechsler D. “Wechsler Memory Scale Manual” San Diego 1987; Psychological Corp., its contents herein considered incorporated by reference.

### Recognitition Test, RAVLT Recall

The recognition test is preferably selected from the group consisting of:

Rey Auditory Verbal Learning test [RAVLT];

### Repeatable Battery for the Assessment of Neuropsychological Status [RBANS];

### California Verbal Learning Test [CVLT]; and

### Consortium to Establish a Registry for Alzheimer Disease Test [CERAD].

Repeatable Battery for the Assessment of Neuropsychological Status (RBANS) is for instance in the RBANS manual by Randolf 1998, can be used to score the recognition performance (RBANS-recognition), and thereby assess or monitor the recognition function. The California Verbal Learning Test (CVLT) is detailed in Delis, D. C., Kramer, J. H., Kaplan, E., & Ober, B. A. (2000), and can be used to score the recognition performance (CVLT-recognition), and thereby assess or monitor the recognition function.

The RAVLT is preferred. In the field, the Rey Auditory Verbal Learning test is subdivided in ‘RAVLT recall’ and RAVLT recognizition’. The RAVLT is a measure of both immediate and delayed episodic verbal learning and memory. The test is ‘episodic’ in that the reading of the word list represents an episode in the subject's memory. The RAVLT is useful in evaluating verbal learning and memory, including proactive inhibition, retroactive inhibition, retention, encoding versus retrieval, and subjective organization. Because the test is brief, straightforward, easy to understand, and appropriate for children, adolescents, and adults ages 7-89 years, it has gained widespread acceptance (source: http://www.annarbor.co.uk/index.php?main_page=indexStcPath=249_{—}303).

For the recall part of the RAVLT, the subject hears a list of words, preferably about 15, and are asked for their immediate recall. This trial is preferably carried out a number of times, preferably about 5 times. Delayed recall of the word list will be examined after an interference period. In the recognition part of the RAVLT, unrelated distracting words are intermixed with the words of the list used in the RAVLT recall part, and the subject is asked which of the words were read aloud before.

For instance, in the immediate recall component (RAVLT-immediate) words are read to the subjects and they are required to recall them as best as they are able after the test administrator has completed reading the list. This first words list (List A), of e.g. 15 words, is read several times, e.g. a total of 3, or 4 or 5 times, and word(s) recall for each of the trials is recorded. It is possible to determine the rate of learning by observing whether there is improved recall with repeated exposure to the words list. After the trials of List A, a second list (List B), of e.g. 15 words, is read to the subjects. Recall for List B is recorded and then the subjects are again asked to recall words from List A. The administration of List B is designed to interfere with recall of List A to make recall more difficult.

For the delayed recall component (RAVLT-delayed), a delay interval, e.g. 10 to 30 minutes, after this last stage of the test, the subjects are asked to recall the words from List A. In the final stage of the test, corresponding to the recognition component (RAVLT-recognition), the subjects are asked to identify List A words from a written list of words. The list is comprised of the original List A words intermingled with new ‘distractor’ words, e.g. the 15 words of list A intermingled with 15 new ‘distractor’ words. Performance on recognition memory tasks typically exceeds that for word recall and so is useful addition in studies of individuals whose memory maybe impaired.

Reference is made to Rey A. “L'examen clinique en psychologie.” Paris: Presses Universitaires de France 1964, its contents herein considered incorporated by reference.

### Wechsler Memory Scale Digit Span Test

In this test, number sequences of increasing length are read to the patient, who is asked to repeat these either forward (Digits Forward) or backward (Digits Backward). The score is derived from the number of sequences correctly recalled.

Reference is made to Wechsler D. “Wechsler Memory Scale Manual” San Diego 1987; Psychological Corp., its contents herein considered incorporated by reference.

### Controlled Word Association Test

The Controlled Word Association Test (COWAT) measures the patient's ability to make verbal associations to specified letters (e.g. C, F and L).

### Category Fluency Test

The Category Fluency test measures the ability to recollect as many words that belong to a certain category, for instance the semantic category ‘animals’. The score is the number of different items belonging to the category named in a predefined time, preferably 60 seconds.

Reference is made to Lezak M D, Howieson D B, Loring D W. “Neuropsychological Assessment” New York: Oxford University Press, 2004, its contents herein considered incorporated by reference.

### Trail Making Test

The NTB preferably comprises at least a Trail Making Test B or ‘TMT-B’. In a further embodiment, the NTB further comprises the Trail Making Test A (‘TMT-A’]. It is particularly preferred to use the Delis Kaplan Executive Function System™ (DKEFS; copyright © 2001 NCS Pearson, Inc.), particularly DKEFS condition 2 and 4 (corresponding to TMT A and B, respectively), most preferably at least DKEFS condition 4.

The TMT was originally developed as part of the Army Individual Test Battery, and is one of the widely used neuropsychological tests for evaluating executive function. The TMT provides information on visual search, scanning, speed of processing, mental flexibility and other executive functions. Briefly, the TMT consists of two parts: TMT-A (‘number sequencing’) requires an individual to draw lines sequentially connecting a number of encircled numbers distributed on a sheet of paper, e.g. 16 to 25. Task requirements are similar for TMT-B (‘number-letter switching’) except that the person must alternate between numbers and letters. The score on each part represents the amount of time to complete the task. More details are provided in T. Tombaugh “Trail Making Test A and B: Normative data stratified by age and education” Archives of Clinical Neuropsychology 19(2004) 203-214.

The TMT-B (more particularly the DKEFS condition 4) has been acknowledged a reliable means for test executive function. Part B of the Trail Making Test is a good general indicator because its cognitive demands include visual scanning, visual-motor coordination and visual-spatial ability adequate enough to understand on an ongoing basis the alternating pattern of numbers and letters. Part B is associated with the processes of distinguishing between numbers and letters, integration of two independent series, ability to learn an organizing principle and apply it systematically, serial retention and integration, verbal problem solving, and planning.

Reference is made to (i) Reitan R M. “The relation of the trail making test to organic brain damage” J Consult Psychol 1955; 19(5):393-394; (ii) Reitan R M. “Validity of the trail making test as an indicator of organic brain damage” Perceptual and Motor Skills 1958; 8:271-276; and (iii) Armitage S G. “An analysis of certain psychological tests used for the evaluation of brain injury” Psychological Monographs 1946; 60(1): no. 277, their contents herein considered incorporated by reference.

### Orientation Task ADAS-Cog Test

This task is designed to determine how well oriented the patient is with regard to time and place. As said before, it is preferred that the NTB and the method according to the present invention do not include the complete ADAS-cog test, but the ADAS-cog orientation task only. It is a concern of the inventors to optimize the training and testing period to periods and frequencies acceptable to the subjects. Hence, in one embodiment, all ADAS-cog tests other than the orientation taks are excluded from the method and NTB of the present invention.

### Letter Digit Substitution Test [LDST]

The Letter Digit Substitution Test is a measure for information processing speed. A number of different letters, preferably about 9, are coupled with other (preferably equal amount of) numbers in a key. Below this key, a random series of letters in cells is presented, and patients are instructed to write down the corresponding digit to the letters as quickly as possible. The score is the number of correctly substituted numbers in a pre-defined time, preferably about 60 seconds.

Reference is made to Rosen W G, Mohs R C, Davis K L. “A new rating scale for Alzheimer's disease” Am J Psychiatry 1984; 141(11):1356-1364, its contents herein considered incorporated by reference.

### Subject

In particular, the subject is a human being that suffers from a neuropsychological disorder, preferably a memory or cognitive disorder, memory decline or cognitive dysfunction, such as Age Associated Memory Impairment (AAMI), multiple sclerosis, vascular dementia, frontotemporal dementia, semantic dementia or dementia with Lewy bodies, and Alzheimer's Disease. The subject is preferably suffering from cognitive dysfunction associated with Alzheimer's disease [AD], Pick's disease, Lewy Body disease, Huntington's disease, or ‘dementia syndrome’. Dementia syndrome encompasses vascular dementia, frontotemporal dementia, semantic dementia.

The subject is preferably a human, preferably an elderly human being, preferably at least 50 years of age. The subject is preferably an AD or dementia patient. In one aspect, the invention is not concerned with the treatment of Alzheimer's disease or dementia itself, but with the treatment of persons suffering from Alzheimer's disease, dementia and/or elderly.

In one embodiment, the subject is preferably a drug-naïve subject, which subject has preferably not been administered any drug for memory improvement and or for AD or dementia at least four weeks prior to the treatment or assessment. Preferably, the term ‘drug naïve’ as used in the present invention refers to subjects who do not ingest one or more of cholinesterase inhibitors, N-methyl-D-aspartate (NMDA) antagonists and ginkgo biloba during treatment or assessment, and preferably have not taken any cognitive ability-affecting drugs in the four weeks prior to the treatment or assessment. The NTB presented here was found very effective in drug naïve subjects.

The NTB according to the invention is particularly useful in monitoring decline in memory and executive functions in cases of mild or prodromal Alzheimer's diseaseHence, in one aspect, the subject is a mild cognitive impairment (MCI) patient (or ‘mild AD patient’ or ‘mild dementia patient’) or an AAMI patient. The patient group may also encompass prodromal patients of neurological disorders, in particular—naïve prodromal AD patients or drug-naïve prodromal dementia patients. A ‘prodromal dementia patient’ is a person who does not suffer from a senile dementia as defined above, but has an increased likelihood to develop senile dementia. Likewise a ‘prodromal Alzheimer patient’ is a person who does not suffer from AD, but has an increased likelihood to develop AD. The diagnostic tools that are used to classify the patients as prodromal patients are available in the art, and for instance summarized in WO 2009/002164, its contents herein incorporated by reference.

In yet a further way of characterizing the subject may be characterized by having a mini-mental state examination [MMSE] of 20-30. The MMSE is a standardized test developed in the art to distinguish between the various (pre-) stages of dementia. It involves a brief 30-point questionnaire that is used to assess cognition. In the time span of about 10 minutes it samples various functions including memory and orientation. The MMSE test includes simple questions and problems in a number of areas: the time and place of the test, repeating lists of words, language use and comprehension, and basic motor skills. Any score of 27 or higher (out of 30) is interpreted as effectively normal; 20-26 indicates mild dementia; 10-19 moderate dementia, and below 10 severe dementia. Copyrights prevent the inventors from including a copy of the questionnaire into the specification, but it is readily accessible on the internet and available through copyright owner Psychological Assessment Resources (PAR). It is first introduced by Folstein et al. (Psych Res 12:189, 1975), and is widely used with small modifications to assess cognition. Preferably, in the present invention, the subjects have a mini-mental state examination (MMSE) of 20-30, more preferably of 20-26, even more preferably a MMSE of 24, 25 or 26. More preferably, the subject having the aforementiond MMSE score range has (or suffers from) Alzheimer's disease, mild cognitive impairment (MCI), age-associated memory impairment (AAMI), multiple sclerosis, vascular dementia, frontotemporal dementia, semantic dementia or dementia with Lewy bodies.

Most preferably, the subjects assessed, monitored or treated in the present invention are drug naïve, and suffer from mild Alzheimer's disease characterized by a MMSE of 20-26, preferably 24-26.

### Product

Throughout the application, the terms ‘product’ and ‘composition’ are used interchangeably and account for the combination of ingredients administered to a subject in need thereof.

In one aspect of the present invention, the composition according to the invention may be used as a pharmaceutical product comprising one or more pharmaceutically acceptable carrier materials.

In another aspect of the present invention, the composition according to the invention may be used as a nutritional product, for example as a nutritional supplement, e.g., as an additive to a normal diet, as a fortifier, to add to a normal diet, or as a complete nutrition.

The pharmaceutical product, preferably for enteral application, may be a solid or liquid galenical formulation. Examples of solid galenical formulations are tablets, capsules (e.g. hard or soft shell gelatine capsules), pills, sachets, powders, granules and the like which contain the active ingredient together with conventional galenical carriers. Any conventional carrier material can be utilized. The carrier material can be organic or inorganic inert carrier material suitable for oral administration. Suitable carriers include water, gelatine, gum Arabic, lactose, starch, magnesium stearate, talc, vegetable oils, and the like. Additionally, additives such as flavouring agents, preservatives, stabilizers, emulsifying agents, buffers and the like may be added in accordance with accepted practices of pharmaceutical compounding. While the individual active ingredients are suitably administered in a single composition, they may also be administered in individual dosage units.

Hence, the invention further relates to a kit of parts comprising i) one or more of uridine and cytidine, or salts, phosphates, acyl derivatives or esters thereof; and ii) a lipid fraction comprising at least one of docosahexaenoic acid (22:6; DHA), eicosapentaenoic acid (20:5; EPA) and docosapentaenoic acid (22:5; DPA), or esters thereof, for the aforementioned use or for use in the aforementioned method. In one embodiment, it is preferred to include iii) choline, or salts or esters thereof.

If the composition is a pharmaceutical product, such product may contain the daily dosage in one or more dosage units. The dosage unit may be in a liquid form or in a solid form, wherein in the latter case the daily dosage may be provided by one or more solid dosage units, e.g. in one or more capsules or tablets.

In another aspect of the present invention, the composition according to the invention may be used in a nutritional product comprising at least one component selected from the group of fats, proteins, and carbohydrates. It is understood that a nutritional product differs from a pharmaceutical product by the presence of nutrients which provide nutrition to the subject to which the composition is administered, in particular the presence of protein, fat, digestible carbohydrates and dietary fibres. It may further contain ingredients such as minerals, vitamins, organic acids, and flavouring agents. Although the term “nutraceutical product” is often used in literature, it denotes a nutritional product with a pharmaceutical component or pharmaceutical purpose. Hence, the nutritional composition according to the invention may also be used in a nutraceutical product.

The product of the invention is an enteral composition, intended for oral administration. It is preferably administered in liquid form. In one embodiment, the product comprises a lipid fraction and at least one of carbohydrates and proteins, wherein the lipid composition provides between 20 and 50 energy % of the food product. In one embodiment, the food product is a liquid composition containing between 0.8 and 1.4 kcal per ml.

Preferably, the composition comprising (i) and (ii) further comprises choline.

Preferably the composition comprising (i) and (ii) further comprises one or more of: phospholipids, vitamin E, vitamin C, selenium, vitamin B12, vitamin B6 and folic acid.

More preferably the composition comprises DHA, EPA, a uridine source (preferably UMP), phospholipids, choline, vitamin E, vitamin C, selenium, vitamin B12, vitamin B6 and folic acid.

### DHA/EPA

The composition comprises at least one ω-3 polyunsaturated fatty acid (LC PUFA; having a chain length of 18 and more carbon atoms) selected from the group consisting of docosahexaenoic acid (22:6; DHA), eicosapentaenoic acid (20:5; EPA) and docosapentaenoic acid (22:5 co-3; DPA), preferably at least one of DHA and EPA. Preferably the present composition contains at least DHA, more preferably DHA and EPA. EPA is converted to DPA (ω-3), increasing subsequent conversion of DPA to DHA in the brain. Hence, the present composition preferably contains a significant amount of EPA, so to further stimulate in vivo DHA formation.

The DHA, EPA and/or DPA are preferably provided as triglycerides, diglycerides, monoglycerides, free fatty acids or their salts or esters, phospholipids, lysophospholipids, glycerol ethers, lipoproteins, ceramides, glycolipids or combinations thereof. Preferably, the present composition comprises at least DHA in triglyceride form.

In terms of daily dosage, the present method preferably comprises the administration of 400 to 5000 mg DHA+EPA+DPA (preferably DHA+EPA) per day, more preferably 500 to 3000 mg (preferably DHA+EPA) per day, most preferably 1000 to 2500 mg (preferably DHA+EPA) per day. DHA is preferably administered in an amount of 300 to 4000 mg per day, more preferably 500 to 2500 mg per day.

The present composition preferably comprises 1-40 wt. % DHA based on total fatty acids, preferably 3-36 wt. % DHA based on total fatty acids, more preferably 10-30 wt. % DHA based on total fatty acids. The present composition preferably comprises 0.5-20 wt. % EPA based on total fatty acids, preferably 2-10 wt. % EPA based on total fatty acids, more preferably 5-10 wt. % EPA based on total fatty acids. The above-mentioned amounts take into account and optimise several aspects, including taste (e.g. too high LCP levels reduce taste, resulting in a reduced compliance).

The present composition preferably contains at least one oil selected from fish oil, algae oil and eggs lipids. Preferably the present composition contains fish oil comprising DHA and EPA.

The ratio of the weights of DHA to EPA is preferably larger than 1, more preferably 2:1 to 10:1, more preferably 3:1 to 8:1. The above-mentioned ratios and amounts take into account and optimise several aspects, including taste (too high LCP levels reduce taste, resulting in a reduced compliance), balance between DHA and precursors thereof to ensure optimal effectiveness while maintaining low-volume formulations.

Sources of DHA possible sources of DHA: tuna oil, (other) fish oils, DHA rich alkyl esters, algae oil, egg yolk, or phospholipids enriched with n-3 LCPUFA e.g. phosphatidylserine-DHA.

The present composition preferably contains a very low amount of arachidonic acid (AA). Preferably the weight ratio DHA/AA in the present composition is at least 5, preferably at least 10, more preferably at least 15, preferably up to e.g. 30 or even up to 60. The present method preferably comprises the administration of a composition comprising less than 5 wt. % arachidonic acid based on total fatty acids, more preferably below 2.5 wt. %, e.g. down to 0.5 wt %.

### ALA/LA

It is preferred that the alpha-linolenic acid [ALA] content of the composition is maintained at low levels. The ALA concentration may preferably be maintained at levels less than 2.0 weight %, more preferably below 1.5 weight %, particularly below 1.0 weight %, calculated on the weight of all fatty acids.

Linoleic acid [LA] concentrations can be maintained at normal levels, i.e. between 20 to 30 weight %, although in one embodiment the LA concentration is also significantly reduced to an amount of <15 g/100 g fatty acids and even less than 10 weight %. The LA concentrations are preferably at least 1 weight % of the fatty acids.

The weight ratio omega-6/omega-3 fatty acids in the present product is preferably below 0.5, more preferably below 0.2, e.g. down to 0.05 or to 0.01. The ratio ω-6/ω-3 fatty acids (C 20 and higher) in the present product is preferably below 0.3, more preferably below 0.15, e.g. down to 0.06 or to 0.03.

### MCT

In one embodiment, the composition contains less than 5 weight %, preferably less than 2 weight % of fatty acids of less than 14 carbon atoms.

Medium chain fatty acids [MCT] are defined to be linear or branched saturated carboxylic acids having six (C6:0), seven (C7:0), eight (C8:0), nine (C9:0) or ten (C10:0) carbon atoms. The amount of MCTs are preferably lower than 2 weight %, more preferably lower than 1.5 weight %, most preferably lower than 1.0 weight % of the total fatty acids. In one embodiment, the sum of the medium chain fatty acids C6:0+C7:0+C8:0 over the sum of C9:0 and C10:0 is less than 2:1, more preferably less than 1.8:1, most preferably less than 1.6:1.

### Saturated and Monounsaturated Fatty Acids

The present composition preferably comprises saturated and/or mono-unsaturated fatty acids. The amount of saturated fatty acids is preferably 6-60 wt. % based on total fatty acids, preferably 12-40 wt. %, more preferably 20-40 wt. % based on total fatty acids. In particular the amount of C14:0 (myristic acid)+C16:0 (palmitic acid) is preferably 5-50 wt. %, preferably 8-36 wt. %, more preferably 15-30 wt. %, based on total fatty acids. The total amount of monounsaturated fatty acids, such as oleic acid and palmitoleic acid, is preferably between 5 and 40 wt. %, more preferably between 15 and 30 wt. %. A composition with these preferred amounts was found to be very effective.

### Uridine, UMP

The present composition comprises uridine, cytidine and/or an equivalent thereof, including salts, phosphates, acyl derivatives and/or esters. In terms of uridine, the composition preferably comprises at least one uridine or an equivalent thereof selected from the group consisting of uridine (i.e. ribosyl uracil), deoxyuridine (deoxyribosyl uracil), uridine phosphates (UMP, dUMP, UDP, UTP), nucleobase uracil and acylated uridine derivatives. In one embodiment, cytidine, CMP, citicoline (CDP-choline) may also be applied. Preferably, the composition to be administered according to the present invention comprises a source of uridine selected from the group consisting of uridine, deoxyuridine, uridine phosphates, uracil, and acylated uridine, and cytidine, more preferably selected from the group consisting of uridine, deoxyuridine, uridine phosphates, uracil, and acylated uridine.

Preferably, the present composition comprises an uridine phosphate selected from the group consisting of uridine monophosphate (UMP), uridine diphosphate (UDP) and uridine triphosphate (UTP); and/or a cytidine phosphate (CMP, CDP, CTP, preferably CMP). Most preferably the present composition comprises UMP, as UMP is most efficiently being taken up by the body. Preferably at least 50 weight % of the uridine in the present composition is provided by UMP, more preferably at least 75 weight %, most preferably at least 95 weight %. Doses that must be administered are given as UMP. The amount of uracil sources can be calculated taking the molar equivalent to the UMP amount (molecular weight 324 Dalton).

The present method preferably comprises the administration of uridine (the cumulative amount of uridine, deoxyuridine, uridine phosphates, nucleobase uracil and acylated uridine derivatives) in an amount of in an amount of 0.08-3 g per day, preferably 0.1-2 g per day, more preferably 0.2-1 g per day. The present method preferably comprises the administration of a composition comprising uridine in an amount of 0.08-3 g UMP per 100 ml liquid product, preferably 0.1-2 g UMP per 100 ml liquid product, more preferably 0.2-1 g per 100 ml liquid product. Preferably 1-37.5 mg UMP per kilogram body weight is administered per day. The above amounts also account for any amounts of cytidine, cytidine phosphates and citicoline incorporated in the composition or method.

Preferably, the present composition comprises uridine phosphate, preferably uridine monophosphate (UMP). The UMP is very efficiently taken up by the body. Hence, inclusion of UMP in the present composition enables a high effectivity at the lowest dosage and/or the administration of a low volume to the subject.

### Choline

In a preferred embodiment, the present composition contains choline, a choline salt and/or choline ester. For the remainder of the paragraph, the term ‘choline’ shall be considered to encompass all these equivalents. The choline salt is preferably selected from choline chloride, choline bitartrate, or choline stearate. The choline ester is preferably selected from a phosphatidylcholine and lyso-phosphatidyl choline. The present method preferably comprises the administration of more than 50 mg choline per day, preferably 80 to 2000 mg choline per day, more preferably 120 to 1000 mg choline per day, most preferably 150 to 600 mg choline per day. The present composition preferably comprises 50 mg to 3000 gram choline per 100 ml of the liquid composition, preferably 200 mg to 1000 mg choline per 100 ml. The above numbers are based on choline, the amounts of choline equivalents or sources can be calculated taking the molar equivalent to choline into account.

### Phospholipids

Preferably, the present composition preferably comprises phospholipids, preferably 0.1-50 wt. % phospholipids based on total weight of lipids, more preferably 0.5-20 wt. %, more preferably between 1 and 10% wt. %, most preferably between 1 and 5 wt. % based on total weight of lipids. The total amount of lipids is preferably between 10 and 30 wt. % on dry matter, and/or between 2 and 10 g lipid per 100 ml for a liquid composition. The composition preferably comprises between 0.01 and 1 gram lecithin per 100 ml, more preferably between 0.05 and 0.5 gram lecithin per 100 ml. A composition with these preferred amounts was found to be very effective.

### Vitamins

The present combination preferably comprises at least one B complex vitamin. The vitamin B is selected from the group of vitamin B1 (thiamine), vitamin B2 (riboflavin), vitamin B3 (niacin or niacinamide), vitamin B5 (pantothenic acid), vitamin B6 (pyridoxine, pyridoxal, or pyridoxamine, or pyridoxine hydrochloride), vitamin B7 (biotin), vitamin B9 (folic acid or folate), and vitamin B12 (various cobalamins). Functional equivalents are encompassed within these terms.

Preferably, at least one vitamin B is selected from the group of vitamin B6, vitamin B12 and vitamin B9. Preferably the present composition comprises at least two selected from the group consisting of vitamin B6, vitamin B12 and vitamin B9. In particular, good results have been achieved with a combination comprising vitamin B6, vitamin B12 and vitamin B9. Again, functional equivalents are encompassed within these terms.

The vitamin B is to be administered in an effective dose, which dose depends on the type of vitamin B used. As a rule of thumb, a suitable minimum or a maximum dose may be chosen based on known dietary recommendations, for instance as recommended by Institute of Medicine (IOM) of the U.S. National Academy of Sciences or by Scientific Committee on Food (a scientific committee of the EU), the information disclosed herein and optionally a limited amount of routine testing. A minimum dose may be based on the estimated average requirement (EAR), although a lower dose may already be effective. A maximum dose preferably does not exceed the tolerable upper intake levels (UL), as recommended by IOM.

If present in the nutritional composition or medicament, the vitamin B6 is usually present in an amount to provide a daily dosage in the range of 0.1 to 100 mg, in particular in the range of 0.5 to 25 mg, more in particular in the range of 0.5 to 5 mg. The present composition preferably comprises 0.1 to 100 mg vitamin B6 per 100 g (liquid) product, more preferably 0.5 to 5 mg vitamin B6 per 100 g (liquid) product, more preferably 0.5 to 5 mg vitamin B6 per 100 g (liquid) product.

If present in the nutritional composition or medicament, the vitamin B12 is usually present in an amount to provide a daily dosage in the range of 0.5 to 15 μg, in particular in the range of 1 to 10 μg, more in particular in the range of 1.5 to 5 μg. The present composition preferably comprises 0.5-15 μg vitamin B12 per 100 g (liquid) product, more preferably 1 to 10 μg vitamin B12 per 100 g (liquid) product, more preferably 1.5 to 5 μg vitamin B12 per 100 g (liquid) product. The term “vitamin B12” incorporates all cobalbumin equivalents known in the art.

Throughout the application, the terms ‘folic acid’, ‘folate’ and ‘B9’ are used interchangeably. If present in the nutritional composition or medicament, the vitamin B9 is usually present in an amount to provide a daily dosage in the range of 50 to 1000 μg, in particular in the range of 150 to 750 μg, more in particular in the range of 200 to 500 μg. The present composition preferably comprises 50 to 1000 μg folic acid per 100 g (liquid) product, more preferably 150 to 750 μg folic acid per 100 g (liquid) product, more preferably 200 to 500 μg folic acid per 100 g (liquid) product. Folates include folic acid, folinic acid, methylated, methenylated and formylated forms of folates, their salts or esters, as well as their derivatives with one or more glutamic acid, and all in either reduced or oxidized form.

### Vitamins C, E

Vitamin C, or a functional equivalent thereof, may be present in an amount to provide a daily dosage in the range of 20 to 2000 mg, in particular in the range of 30 to 500 mg, more in particular in the range of 75 to 150 mg. In one embodiment, vitamin C, or a functional equivalent thereof, is present in an amount in the range of 20 to 2000 mg, in particular in the range of 30 to 500 mg, more in particular in the range of 75 to 150 mg per 100 ml of the composition.

Tocopherol and/or an equivalent thereof (i.e. a compound having vitamin E activity) may be present in an amount to provide a daily dosage in the range of 10 to 300 mg, in particular in the range of 30 to 200 mg, more in particular in the range of 35 to 100 mg, to prevent oxidative damage resulting from dietary PUFA. In one embodiment, tocopherol and/or equivalent is present in an amount in the range of 10 to 300 mg, in particular in the range of 30 to 200 mg, more in particular in the range of 35 to 100 mg per 100 ml of the composition. The term “tocopherol and/or an equivalent thereof”, and ‘alpha-TE’, as used in this description, comprises tocopherols, tocotrienols, pharmaceutical and/or nutritional acceptable derivatives thereof and any combination thereof. The above numbers are based on tocopherol equivalents, recognized in the art.

Selenium The present composition preferably contains selenium, because of its antioxidant activity. Preferably the present method provides the administration of a composition comprising 0.01 and 5 mg selenium per 100 ml liquid product, preferably 0.02 and 0.1 mg selenium per 100 ml liquid product. The amount of selenium administered per day is preferably more than 0.01 mg, more preferably 0.01 to 0.5 mg.

### Protein

Although the composition may further comprise proteinaceous material, it has been found that such component is not deemed necessary. In fact, it is thus possible to concentrate the actives in a low volume composition. Should a protein fraction be included, the protein fraction comprises intact proteins, peptides as may be obtained by hydrolyses of intact proteins and by syntheses, derivatives of peptides comprising more than 80 weight % amino acids. Nitrogen from nucleosides material and choline will not be calculated as being protein.

In one embodiment, it is preferred that the amount of taurine (including taurine salts) is less than 0.1 g, preferably less than 0.05 g per daily dose. Additionally or alternatively, it is preferred that the amount of taurine (including taurine salts) is less than 5 mg, more preferably less than 2.5 g per 100 g composition.

In one embodiment, the composition comprises less than 25 mg, more preferably less than 20 mg, most preferably less than 15 mg cysteine and taurine per 100 ml of the (liquid) composition. In one embodiment, the composition comprises less than 25 mg, more preferably less than 20 mg, most preferably less than 15 mg cysteine per 100 ml of the (liquid) composition. It is preferred that the protein fraction comprises more than 70 weight % of casein or caseinates, or hydolysates thereof, and more preferably 80 weight % or more, because caseins comprise relatively low amounts of cysteine compared to other protein sources. It is further preferred to heat the liquid composition in order to oxidize the cysteine molecules present in the protein. This impairs biological availability of any residual cysteine as present in the formula. A preferred heat treatment involves sterilization. It is preferred to maintain the temperature remains below 135° C., preferably less than 132° C. combined with a sufficient long time to have the cysteine oxidized, i.e. more than 30 seconds, preferably more than 40 seconds.

In one embodiment, it is preferred that the composition has a protein content of less than 15 en %, more preferably less than 10 en %, most preferably less than 5 en % of the total energy content of the composition. The energy percentages of the components are calculated using the calculation factors 9 kcal per g lipid, 4 kcal per g protein or g digestible carbohydrates, 2 kcal per g dietary fibers and zero kcal for the other components in the composition. In one embodiment, it is preferred that the composition comprises less than 0.5 to 10 g protein per 100 ml, more preferably less than 1 to 6 gram protein per 100 ml, most preferably 2 to 6 gram protein/100 ml.

A preferred composition according to the invention comprises, per daily dose or per 100 ml composition:
100-500 mg, preferably 200-400 mg EPA,
900-1500 mg, preferably 950-1300 mg DHA,
50-600 mg, preferably 60-200 mg phospholipids,
200-600 mg, preferably 300-500 mg choline,
400-800 mg, preferably 500-700 mg UMP (uridine monophosphate),
20-60 mg, preferably 30-50 mg vitamin E (alpha-TE),
60-100 mg, preferably 60-90 mg vitamin C,
40-80 μg, preferably 45-65 μg selenium,
1-5 μg, preferably 2-4 μg vitamin B12,
0.5-3 mg, preferably 0.5-2 mg vitamin B6, and
200-600 μg, preferably 300-500 μg folic acid.

More preferred, a composition according to the invention comprises per 100 ml composition:
100-500 mg, preferably 200-400 mg EPA,
900-1500 mg, preferably 950-1300 mg DHA,
50-600 mg, preferably 60-200 mg phospholipids,
200-600 mg, preferably 300-500 mg choline,
400-800 mg, preferably 500-700 mg UMP (uridine monophosphate),
20-60 mg, preferably 30-50 mg vitamin E (alpha-TE),
60-100 mg, preferably 60-90 mg vitamin C,
40-80 rig, preferably 45-65 μg selenium,
1-5 μg, preferably 2-4 μg vitamin B12,
0.5-3 mg, preferably 0.5-2 mg vitamin B6, and
200-600 μg, preferably 300-500 μg folic acid.

The compositions as described above can be used as a nutritional therapy, nutritional support, as a medical food, as a food for special medical purposes or as a nutritional supplement. Such product can be consumed at one, two or three servings between 75 and 200 ml per day or per unit, most preferably between 90 and 150 ml/day, most preferably about 125 mL per day in the aforementioned applications.

The subjects that can benefit from the method and composition of the invention often experience problems with eating. Their sensory capabilities and/or control of muscles can become imparted, as well as in some instances their ambition to apply proper eating habits. Swallowing and/or mastication may be problematic. Hence, the present composition is preferably provided in the form of a drink capable of being ingested through a straw.

Related therewith, the composition according to the invention preferably has a low viscosity, preferably a viscosity between 1 and 2000 mPa·s measured at a shear rate of 100 sec-1 at 20° C., more preferably a viscosity between 1 and 100 mPa·s measured at a shear rate of 100 sec-1 at 20° C. In a preferred embodiment the present composition has a viscosity of 1-80 mPas at a shear rate of 100 per sec at 20° C., more preferably of 1-40 mPas at a shear rate of 100 per sec at 20° C. These viscosity measurements may for instance be performed using plate and cone geometry.

To be optimally accepted by the subject, the present composition preferably has an osmolality of 300 to 800 mOsm/kg. However, the energy density of the product is preferably not so high that it interferes with normal eating habits. When in liquid form, the present product preferably contains between 0.2 and 3 kcal/ml, more preferably between 0.5 and 2, between 0.7 and 1.5 kcal/ml.

### Computer System

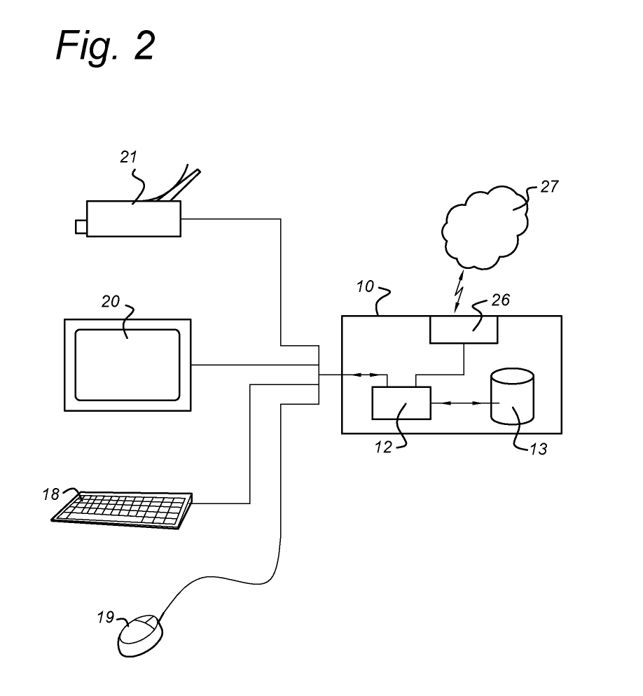

Method steps as described above may be performed by a computer system 10 or by two or more cooperating computer systems 01. FIG. 2 shows a schematic block diagram of an embodiment of a computer system 10.

The computer system 10 comprises a processor unit 12 for performing arithmetical operations. The processor unit 12 is connected to memory unit 13 that stores instructions and data, such as a hard disk, a Read Only Memory (ROM), Electrically Erasable Programmable Read Only Memory (EEPROM) and a Random Access Memory (RAM).

The processor unit 12 is arranged to read and execute instructions from the memory unit 13 to enable the processor unit 12 to perform one or more of the method steps described.

The steps may comprise user-interaction using one or more input devices, such as a keyboard 18 and a mouse 19, one or more output devices, such as a display 20 and a printer 21. The processor unit 12 is also arranged to write data to the memory unit 13 for storage. These data may relate to (intermediate) results of the method steps.

In an embodiment of the invention the computer system 10 comprises a database stored in said memory unit 13 containing information or instructions about one or more, preferably all of the individual tests, and information on calculating the scores from these tests.

The computer system 10 shown in FIG. 2 also comprises an input output device (I/O) 26 that is arranged to communicate with other computer systems (not shown) via a communication network 27.

However, it should be understood that there may be provided more and/or other memory units, input devices and read devices known to persons skilled in the art. Moreover, one or more of them may be physically located remote from the processor unit 12, if required. The processor unit 12 is shown as one box, however, it may comprise several processing units functioning in parallel or controlled by one main processor unit that may be located remote from one another, as is known to persons skilled in the art.

It is observed that, although all connections in FIG. 2 are shown as physical connections, one or more of these connections can be made wireless. They are only intended to show that “connected” units are arranged to communicate with one another in someway.

In one aspect, the invention pertains to a method for improving the global or composite NTB score of a subject in need thereof, comprising administering to said subject the composition comprising the aforementioned components (i)-(ii), and as further characterized here above. The NTB is characterized in the text above.

In one aspect, the invention pertains to a method for treating a subject in need thereof, administering, preferably at least daily, to said subject a composition comprising the aforementioned components (i)-(ii), and as further characterized here above; and monitoring said subject to an NTB test such as characterized above, and calculating the global or composite NTB score from the individualized tests comprised in the NTB.

In a further aspect, the invention pertains to a composition for use in for improving the global or composite NTB score of a subject in need thereof, wherein said composition comprises (i)-(ii), and as further characterized here above; and monitoring said subject to an NTB test preferably as defined herein.

In one aspect, the invention pertains to a method for assessing cognition of a subject using the NTB as detailed here above. The method may comprise administering said subject with a composition designed or suited for improving cognitive function, said composition preferably comprising (i)-(ii) as defined herein.

In a further aspect, the invention pertains to a composition for improving cognitive function, for use in assessing cognitition of a subject in need thereof, wherein cognition may be assessed or monitored using the NTB as detailed here above.

In yet another aspect, the invention pertains to the use of a composition in the manufacture of a product for improving cognitive function of a subject in need thereof, wherein cognition of said subject is assessed or monitored using the NTB as detailed here above, and said composition preferably comprising (i)-(ii) as defined herein.

### EXAMPLES

### Example 1

### Packaged Composition for Comprising Per 125 ml

Energy 125 kcal; Protein 3.9 g; Carbohydrate 16.5 g; Fat 4.9 g.

Fat includes 1.5 g DHA+EPA, and 106 mg phospholipids (soy lecithin); Choline 400 mg; UMP (uridine monophosphate) 625 mg; Vitamin E 40 mg alpha-TE; Vitamin C 80 mg; Selenium 60 μg; Vitamin B12 3 μg; Vitamin B6 1 mg; Folic acid 400 μg.

Minerals and trace elements: Sodium 125 mg; Potassium 187.5 mg; Chloride 156.3 mg; Calcium 100 mg; Phosphorus 87.5 mg; Magnesium 25 mg; Iron 2 mg; Zinc 1.5 mg; Copper 225 rig; Manganese 0.41 mg; Molybdenum 12.5 rig; Chromium 8.4 rig; Iodine 16.3 μg. Vitamins: Vit. A 200 μg-RE; vit. D3 0.9 rig; vit. K 6.6 rig; Thiamin (B1) 0.19 mg; Riboflavin (B2) 0.2 mg; Niacin (B3) 2.25 mg-NE; Pantothenic acid (B5) 0.66 mg; Biotin 5 μg.

### Example 2

### Clinical Study

A proof-of-concept study in drug-naïve patients with mild AD (MMSE 20-26) showed that a composition according to the invention (see table 1; comprising DHA, EPA, UMP, choline, phospholipids, vitamins B6, B9, B12, vitamins C and E, Selenium) taken once per day was safe and improved delayed recall function of a subject after 12 weeks, the co-primary endpoint of the study. Source: Scheltens et al., “Efficacy of a medical food in mild Alzheimer's disease: A randomized controlled trial” Alzheimer's & Dementia 6 (2010), 1-10.

TABLE 1
Nutritional composition used in clinical trials

| component | Amount per daily dose | |
|---|---|---|
| EPA | 300 | mg |
| DHA | 1200 | mg |
| Phospholipids | 106 | mg |
| Choline | 400 | mg |
| UMP | 625 | mg |
| Vitamin E (alpha-TE) | 40 | mg |
| Vitamin C | 80 | mg |
| Selenium | 60 | μg |
| Vitamin B12 | 3 | μg |
| Vitamin B6 | 1 | mg |
| Folic acid | 400 | μg |

*125 ml, daily dose.

The present study was designed to confirm the effect of the composition on memory in drug-naïve patients with mild AD, and also to extend the investigation through a longer intervention period of 24 weeks and through utilization of the whole memory domain z-score of a Neuropsychological Test Battery (NTB). Secondary objectives were to investigate safety and tolerance of the intervention, and to assess the effects on executive function (z score) (TMTs, CF, COWAT, digit span), total composite score NTB and individual items NTB.

### Material and Methods

The study was a randomized, controlled, double-blind study, conducted at 27 study centers in six European countries (the Netherlands, Germany, France, Belgium, Italy and Spain). Drug-naïve patients with mild AD (MMSE scores 20) and diagnosis of probable AD according to the NINCDS-ADRDA criteria, were randomly assigned (1:1) to the composition including the components according to table 1, or an iso-caloric control product. The duration of intervention was 24 weeks.

The memory domain score of a Neuropsychological Test Battery (NTB) was the primary outcome parameter. This memory composite score was derived from the Rey Auditory Verbal Learning Test (RAVLT: immediate recall, delayed recall and recognition performance) and the Wechsler Memory Scale (WMS) verbal paired associates test (immediate and delayed recall).

Secondary outcomes resulting from the NTB were the executive function domain, total composite score and individual item scores. The other NTB items were WMS Digit Span, Trail Making Tests part A and B, Category Fluency, Controlled Word Association Test, the ADAS-cog orientation task and the Letter Digit Substitution Test. Other secondary outcome parameters were the Disability Assessment for Dementia (DAD) scale, EEG (basic frequency and functional connectivity analysis), product compliance, tolerance and safety. Main study parameters were assessed at baseline, week 12 and week 24. For the statistical analysis of the data, a repeated measures mixed model was used. The trial was registered with the ICMJE compliant www.trialregister.nl (NTR1975).

### Results

A total of 259 drug-naïve subjects were randomized (2.6% screen failures). In the overall study population no differences in baseline characteristics were noted between the study groups. The mean age was 73.8 (±7.7) years, the mean screening MMSE was 25.0 (±2.8) and 51% were male. A pre-specified blinded interim analysis was conducted to check whether the calculated sample size was adequate and that no safety concerns had arisen, and the independent Data Monitoring Committee recommended continuation of the trial without modification. From the 259 subjects randomized, 238 subjects (91.9%) completed the study. Five subjects did not complete the study because of a (serious) adverse event ((S)AE); 3 in the group having received the composition and 2 in the control group, and no differences between study groups were noted in the occurrence of (S)AEs. No clinically relevant differences in blood safety parameters were noted. The average compliance during 24 weeks was very high at 97% and not different between the groups. High compliance was confirmed by marked and significant changes in (nutritional) biomarkers of compliance, e.g. docosahexaenoic acid in erythrocyte membranes and plasma homocysteine.

During 24 weeks, the composition of the invention significantly improved the primary endpoint memory performance (composite memory domain z-score resulting from the NTB) compared to control product (repeated measures mixed model, p=0.025). The significant effect on memory performance was confirmed by individual tasks of the NTB memory domain.

The results in terms of the composite NTB score are shown in FIGS. 3 and 4. In FIG. 3 the composite score also includes the contributions from the ADAS cog orientation task, and the letter digit substitution test.

### DISCUSSION

In conclusion, this study showed that 24-weeks of supplementation with the active composition improved the composite NTB score, and is well-tolerated in drug-naïve patients with mild AD.

1-20. (canceled)

21. A method for improving the composite Neuropsychological Test Battery (NTB) score of a subject in need thereof, the method comprising administering to the subject a composition comprising:
i) one or more of uridine and cytidine, or salts, phosphates, acyl derivatives or esters thereof; and
ii) a lipid fraction comprising at least one of docosahexaenoic acid (22:6; DHA), eicosapentaenoic acid (20:5; EPA) and docosapentaenoic acid (22:5; DPA), or esters thereof.

22. The method according to claim 21, wherein the subject suffers from a memory or cognitive disorder, memory decline or cognitive dysfunction, such as Age Associated Memory Impairment (AAMI), Alzheimer's Disease, multiple sclerosis, vascular dementia, frontotemporal dementia, semantic dementia or dementia with Lewy bodies.

23. The method according to claim 21, wherein the subject suffers from Alzheimer's Disease or dementia syndrome, including mild or prodromal AD or dementia.

24. The method according to claim 21, wherein the subject has a mini-mental state examination (MMSE) of 20-30.

25. The method according to claim 21, wherein the composition comprises 200-600 mg choline, or salts or esters thereof, per 100 ml composition.

26. The method according to claim 21, wherein the composition comprises at least one B vitamin selected from the group consisting of vitamin B6, vitamin B12 and vitamin B9.

27. The method according to claim 21, wherein the composition comprises, per 100 ml composition, at least 500 mg of DHA and at least 50 mg of uridine.

28. The method according to claim 21, wherein the composition comprises, per 100 ml composition:
(a) 50-1000 mg phospholipids,
(b) 0.5-3 mg vitamin B6,
(c) 50-500 μg folic acid, and
(d) 1-30 μg vitamin B12.

29. The method according to claim 21, wherein the composition comprises, per 100 ml composition:
(a) 100-500 mg EPA,
(b) 1000-1500 mg DHA,
(c) 50-600 mg phospholipids,
(d) 200-600 mg choline,
(e) 400-800 mg uridine monophosphate,
(f) 20-60 mg vitamin E,
(g) 60-100 mg vitamin C,
(h) 40-80 μg selenium,
(i) 1-5 μg vitamin B12,
(j) 0.5-3 mg vitamin B6, and
(k) 200-600 μg folic acid.

30. The method according to claim 21, wherein the NTB comprises at least one of:
a. Wechsler memory scale (WMS)—verbal paired associates test;
b. Rey Auditory Verbal Learning test recall (RAVLT recall);
c. Recognition test, preferably selected from the group consisting of:
i. Rey Auditory Verbal Learning test (RAVLT recognition);
ii. Repeatable Battery for the Assessment of Neuropsychological Status (RBANS);
iii. California Verbal Learning Test (CVLT); and
iv. Consortium to Establish a Registry for Alzheimer Disease Test (CERAD);
d. WMS digit span test;
e. Controlled Word Association test (COWAT);
f. Category fluency test;
g. Trail Making Test (TMT);
h. Orientation task ADAS-cog test; and
i. Letter digit substitution test.

31. The method according to claim 30, wherein the RAVLT recall comprises a RAVLT immediate recall test, a RAVLT delayed recall test, or both.

32. The method according to claim 31, wherein the NTB comprises (b) RAVLT immediate recall and RAVLT delayed recall, and (c) RAVLT recognition.

33. The method according to claim 30, wherein the NTB comprises:
WMS VPA immediate test;
WMS VPA delayed test;
RAVLT immediate recall test;
RAVLT delayed recall test;
RAVLT recognition test;
and
WMS digit span test;
COWAT test;
Category fluency test;
TMT;
and optionally:
Orientation task ADAS-cog test; and Letter digit substitution test.

34. A method for assessing cognition of a subject, comprising subjecting the subject to:
a. Wechsler memory scale (WMS)—verbal paired associates test;
b. Rey Auditory Verbal Learning test recall (RAVLT recall);
c. Recognition test, preferably selected from the group consisting of
i. Rey Auditory Verbal Learning test (RAVLT recognition);
ii. Repeatable Battery for the Assessment of Neuropsychological Status (RBANS);
iii. California Verbal Learning Test (CVLT); and
iv. Consortium to Establish a Registry for Alzheimer Disease Test (CERAD);
d. WMS digit span test;
e. Controlled Word Association test (COWAT);
f. Category fluency test;
g. Trail Making Test (TMT);
h. Orientation task ADAS-cog test; and
i. Letter digit substitution test,
and optionally administering the subject with a composition comprising:
(i) one or more of uridine and cytidine, or salts, phosphates, acyl derivatives or esters thereof; and
(ii) a lipid fraction comprising at least one of docosahexaenoic acid (22:6; DHA), eicosapentaenoic acid (20:5; EPA) and docosapentaenoic acid (22:5; DPA), or esters thereof.

35. The method according to claim 34, wherein the RAVLT recall comprises a RAVLT immediate recall test, a RAVLT delayed recall test, or both.

36. The method according to claim 34, wherein the NTB comprises a (b) RAVLT immediate recall, RAVLT delayed recall and a (c) RAVLT recognition test.

37. The method according to claim 34, further comprising calculating memory function and executive function domain scores and/or calculating a composite score from the data retrieved for the individual tests (a)-(i).

38. The method according to claim 37, further comprising comparing the data retrieved for the individual test scores (a)-(i), the calculated memory function and executive function domain scores and/or the calculated composite score at various points in time.

39. A method for treating a subject, comprising:
(a) subjecting a patient to an NTB; and
(b) administering to the subject a composition comprising:
i) one or more of uridine and cytidine, or salts, phosphates, acyl derivatives or esters thereof; and
ii) a lipid fraction comprising at least one of docosahexaenoic acid (22:6; DHA), eicosapentaenoic acid (20:5; EPA) and docosapentaenoic acid (22:5; DPA), or esters thereof.

40. The method according to claim 39, further comprising calculating a composite score from the data retrieved for the individual tests of the NTB.
